(19) United States
(12) Patent Application Publication
(10) Pub. No.: US 2005/0095267 A1
(43) Pub. Date: May 5, 2005

(54) Nanoparticle-based controlled release polymer coatings for medical implants

(76) Inventors: Todd Campbell, Petaluma, CA (US); Kishore Udipi, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/727,894

(22) Filed: Dec. 3, 2003

Related U.S. Application Data
(60) Provisional application No. 60/431,045, filed on Dec. 4, 2002.

(51) Int. Cl. A61F 2/06; A61K 9/16; A61K 9/50

(52) U.S. Cl. 424/425; 623/1.42; 424/490

(57) ABSTRACT

Implantable medical devices having a nanoparticle coating applied thereon. The nanoparticle coating comprises nanopulverized antiproliferative compounds. More specifically, the nanoparticulate antiproliferative compounds comprise particles less than 500 nm in size. The nanoparticle size of the compounds improves compounds' solubility. In addition to improving the solubility of otherwise insoluable antiproliferative compounds, the nanoparticle size minimizes the preparation and formulation required to prepare a dose of the antiproliferative compounds.

### RELATED APPLICATIONS

This application claims priority to U.S. provisional patent application Ser. No. 60/831,045 filed Dec. 4, 2002 the entire contents of which are incorporated herein in their entirety.

### FIELD OF THE INVENTION

The invention relates controlled release coatings for implantable medical devices. Specifically, the present invention relates to controlled release coatings for implantable vascular devices and methods for making same.

### BACKGROUND OF THE INVENTION

Drug releasing medical devices are desirable as a wide variety of drugs can be associated with or applied to the surface of the medical devices and subsequently released from the surface of the device after implantation of the device within the patient's body. For example, the surfaces of a catheter can be coated with antibiotics in order to prevent bacterial infection at the implantation site. Other drug-releasing medical implants include, for example, drug-releasing stents. These stents have been particularly useful because they not only provide the mechanical structure to maintain damaged blood vessel patency, but they may also release drugs into the surrounding tissue to prevent the re-narrowing of the blood vessel.

However, there remain challenges to effectively control drug delivery to the site of disease or injury via drug-releasing medical implants. Generally, drug therapies associated with medical implants released from the medical implants by diffusion. Alternatively, the drug therapies are released from the medical implants via bulk erosion. That is, those drug therapies that are delivered to the site of implantation by a polymeric coating are released as the polymeric coating is physically or chemically eroded. Thus, given these drug-releasing mechanisms, the drug therapies are released soon after implantation of the medical implant. While these and other methods of drug delivery have proven useful, there still remains a need for controllably releasing drug therapies to a site of injury or disease via drug-releasing medical implants.

### BRIEF SUMMARY OF THE INVENTION

The present invention provides methods and compositions for controlled drug release rate and kinetics using a combination the drug nano-particle size, nano-particle size distribution, drug-in-polymer solubility, the polymers' stability (non-bioabsorbable versus bioaborbable), coating thickness, the number of and the presence or absence of polymer layers, polymer primer coats and polymer caps coats. The present invention provides nanoparticle coatings, medical implants having nanoparticle coatings, and methods for their manufacture and use. In a broad aspect, the various embodiments of the nanoparticle coatings of the present invention comprise nanopulverized drug compounds. In a broad aspect, the nanoparticulate compounds comprise particles less than 500 nm in size. As those skilled in the art will appreciate, the smaller size of the compounds improves the solubility of these compounds as well as provides various benefits disclosed herein. For example, prolonged release of the drug compounds can be achieved as a result of the increased surface area of these compounds from nanopulverization. Furthermore, the smaller size of the drug compounds can improve the distribution of the compounds on the surface of a medical device.

In one exemplary embodiment of the present invention, the nanoparticle coating comprises one or more anti-proliferative compounds having particle sizes ranging from approximately 10 nanometers (nm) approximately 1000 nm. In another embodiment, the drug coating comprises compounds having substantially similar particle sizes.

Another aspect of the various embodiments of the present invention, the nanoparticle coatings can be applied to a wide variety of medical implants including, but not limited to, stents, catheters, micro-particles, probes, vascular grafts, access devices, indwelling access ports, valves, plates, barriers, supports, shunts, discs, joints, as well as virtually any device intended to spend time with a patient's body or vasculature. Additionally, the present invention is directed to methods of making medical implants having the nanoparticle coatings of the present invention applied thereon. According to one exemplary method, the drug compounds of the present invention are nanopulverized by any processes known or developed in the art. Nanopulverization refers generally to processes used to produce the nanoparticle compounds as disclosed herein. For example, nanopulverization can comprise mechanical, chemical and physical processes that produce nanoparticle sizes ranging from approximately 10 nm to approximately 1000 nm. The nanopulverized compounds are then directly applied to at least one surface of a medical implant. According to another exemplary method, the nanopulverized compounds are suspended in a polymer matrix (for example, and not intended as a limitation, the matrix includes a terpolymer-bipolyer blend). The polymer matrix can then be applied to at least one surface of the medical implant.

Additionally, the present invention is directed to methods for inhibiting the proliferation of cells surrounding a medical implant. In one exemplary method, the medical implants having the nanoparticle coatings of the present invention applied thereon are delivered to a site of injury or disease. According to one method, drug compounds are released from the surface of the medical implant. Alternatively, in another exemplary method, the drug compounds are suspended in a matrix having openings of similar or variable sizes. The release of the drug compounds from this matrix can be regulated by the size of the drug compounds and/or the size of the openings through which the drug compounds travel through.

### BRIEF DESCRIPTION OF THE FIGURES

FIG. 1 graphically depicts idealized first-order kinetics associated with drug release from a polymer coating.

FIG. 2 graphically depicts idealized zero-order kinetics associated with drug release from a polymer coating.

### DEFINITION OF TERMS

Prior to setting forth the invention, it may be helpful to an understanding thereof to set forth definitions of certain terms that will be used hereinafter:

Animal: As used herein “animal” shall include mammals, fish, reptiles and birds. Mammals include, but are not limited to, primates, including humans, dogs, cats, goats, sheep, rabbits, pigs, horses and cows.

Bioactive agent: As used herein “bioactive agent” shall included drugs such as anti-proliferative compounds, cytostatic compounds, toxic compounds, anti-inflammatory compounds, analgesics, antibiotics, protease inhibitors, statins, nucleic acids, polypeptides, and delivery vectors including recombinant micro-organisms, liposomes, the like (see Drugs below).

Biocompatible: As used herein “biocompatible” shall mean any material that does not cause injury or death to the animal or induce an adverse reaction in an animal when placed in intimate contact with the animal's tissues. Adverse reactions include inflammation, infection, fibrotic tissue formation, cell death, or thrombosis.

Bioabsorbable: As used herein “bioabsorable” means a polymer that is absorbed by the body and broken down into non-toxic metabolites such as lactic acid, carbon dioxide or simple sugars. Furthermore, bioerodable, biooresorbable and bioabsorbable shall be used as synonyms without distinction.

Controlled release: As used herein “controlled release” refers to the release of a bioactive compound from a medical device surface at a predetermined rate. Controlled release implies that the bioactive compound does not come off the medical device surface sporadically in an unpredictable fashion and does not “burst” off of the device upon contact with a biological environment (also referred to herein a first order kinetics) unless specifically intended to do so. However, the term “controlled release” as used herein does not preclude a “burst phenomenon” associated with deployment. In some embodiments of the present invention an initial burst of drug may be desirable followed by a more gradual release thereafter. The release rate may be steady state (commonly referred to as “timed release” or zero order kinetics), that is the drug is released in even amounts over a predetermined time (with or without an initial burst phase) or may be a gradient release. A gradient release implies that the concentration of drug released from the device surface changes over time.

Compatible: As used herein “compatible” refers to a composition possess the optimum, or near optimum combination of physical, chemical, biological and drug release kinetic properties suitable for a controlled release coating made in accordance with the teachings of the present invention. Physical characteristics include durability and elasticity/ductility, chemical characteristics include solubility and/or miscibility and biological characteristics include biocdmpatibility. The drug release kinetic should be either near zero-order or a combination of first and zero-order kinetics.

Copolymer: As used here in a “copolymer” will be defined as ordinarily used in the art of polymer chemistry. A copolymer is a macromolecule produced by the simultaneous or step-wise polymerization of two or more dissimilar units such as monomers. Copolymer shall include bipolymer (two dissimilar units) terpolymer (three dissimilar units) etc.

Drug(s): As used herein “drug” shall include any bioactive agent having a therapeutic effect in an animal. Exemplary, non limiting examples include anti-proliferatives including, but not limited to, macrolide antibiotics including FKBP 12 binding compounds, estrogens, chaperone inhibitors, protease inhibitors, protein-tyrosine kinase inhibitors, peroxisome proliferator-activated receptor gamma ligands (PPARγ), hypothemycin, nitric oxide, bisphosphonates, epidermal growth factor inhibitors, antibodies, proteasome inhibitors, antibiotics, tubulin-microtubule inhibitors (cytoskeleton inhibitors) such as but not limited to epothilones and taxol, anti-sense nucleotides and transforming nucleic acids.

Ductility: As used herein “ductility, or ductile” is a polymer attribute characterized by the polymer's resistance to fracture or cracking when folded, stressed or strained at operating temperatures. When used in reference to the polymer coating compostions of the present invention the normal operating temperature for the coating will be between room temperature and body temperature or approximately between 15° C. and 40° C. Polymer durability in a defined environment is often a function of its elasticity/ductility.

Glass Transition Point: As used herein “glass transition point” or “Tg” is the temperature at which an amorphous polymer becomes hard and brittle like glass. At temperatures above its Tg a polymer is elastic or rubbery; at temperatures below its Tg the polymer is hard and brittle like glass. Tg may be used as a predictive value for elasticity/ductility.

Homopolymer: As used herein “homopolymer” shall mean a polymer being composed of a single monomer.

Hydrophillic: As used herein in reference to the bioactive agent, the term “hydrophilic” refers to a bioactive agent that has a solubility in water of more than 200 micrograms per milliliter.

Hydrophobic: As used herein in reference to the bioactive agent the term “hydrophobic” refers to a bioactive agent that has a solubility in water of no more than 200 micrograms per milliliter.

Nano-particulate or nano-particle: As used here in a nano-particulate or nano-particle is defined as a milled solid, preferably an therapeutic, that has been reduced to a size most conveniently measured in nanometers. The size of the particle contributes to its controlled release as follows. The smaller the particle, the more surface area of drug that is exposed to the physiological environment per unit measure of total drug. For example, 50 mg of epothilone is applied to a vascular sent dispersed in a terpolymer; co-polymer blend such as those discussed below. Given that the polymer's control over elusion rate remains constant regardless of particle size, if the 50 mg of epotilone is milled to form 10 nm sized particles, vastly more surface area will be exposed to the physiological environments, and the encasing polymer, than the same 50 mg milled to 500 nm sized particles. Thus the elusion rate of the 10 nm milled epothilone will exceed that of the 500 nm milled epothilone.

Polymer subunits: As used herein “polymer subunit” or “subunit” refers to the polymer's individual molecular building blocks. In homoploymers the subunits are identical monomers such as (poly)ethylene or (poly)styrene. However, copolymers can have numerous possible configurations. Bipolymers are the simplest copolymer and will be used in the following example. Bipolymers are composed of two dissimilar subunits. The subunits can be separate monomers, or oligomers. For example, a bipolymer having monomeric subunits is composed of two monomers such as ethylene (E) and styrene (S). The polymer chain can be random (for example, DNA and polypeptides are quintessential random polymers), non-random (also referred to occasionally as step growth polymers) blocked or segmented. In random bipolymers, as the name implies, there is no defined order to the monomer sequence, for example --EESESSEESSES-- (of course reaction kinetics may favor one coupling reaction over another; these examples are merely for illustrative purposes). Non-random bipolymers would have an alternating configuration such as ---ESESESESESESESES--. Block copolymers have a high number of covalently bonded repeat subunits such as -EEEEEEEESSSSSSSSSEEEEEEEEE—(ABA configuration) or -EEEEEEEEEEESSSSSSSSSSS—(an ABn configuration). Finally, segmented bipolymers have a small number of repeat subunits such as --EESSEESSEESS--. If a third polymer is added, a terpolymer results. For example, say acrylic acid is added (A). A random terpolymer would look like --AAESASSEAEESAAESEASEASEA--. A non-random terpolymer would look like --ASEASEASEASEASEASEASE--. And a block terpolymer may look like this --AAASSSEEEAAASSSEEEAAASSSEEE--. There are myriad other possible configurations depending on the number of monomeric subunits involved. Still more complex copolymers are possible when the subunits are polymers themselves (oligomeric subunits). Copolymer and terpolymers composed of oligomeric subunits often resemble random and block polymers in their behavior and therefore will not be considered further. Finally, this brief description of polymer primary structure (the chain makeup) did not consider graft polymers (where monomer and polymer side chains are attached as pendent groups to the primary polymer chain) or crosslinking between chains and/or pendent groups (secondary polymer structure). However, any and all of the primary and secondary structures discussed herein and variations thereon are considered within the scope of the present invention.

Units of Measure: As used herein solubility parameters for polymers and solvents will be expressed in δ as originally defined by Hildebrand and Hansen (see for example Properties of Polymers. 1990. 3^{rd} edition. D. W. van Krevelen. Elsevier press. ISBN 0-444-88160-3. The entire contents or which are herein incorporated by reference). δ is a thermodynamic unit expressed in J^{1/2}/cm^{3/2}. However, the reader is cautioned that beginning in 1984 a new value for δ has been adopted and designated δ (SI) and expressed in MPa^{1/2}. To covert between δ (J^{1/2}/cm^{3/2}) and δ(SI) (MPa^{1/2}) multiply δ by 2.0045 or divide δ(SI) by 0.488.

### DETAILED DESCRIPTION OF THE INVENTION

The present invention provides methods and compositions for controlled drug release rate and kinetics using a combination the drug nano-particle size, nano-particle size distribution, drug-in-polymer solubility, the polymers' stability (non-bioabsorbable versus bioaborbable), coating thickness, the number of and the presence or absence of polymer layers, polymer primer coats and polymer caps coats. The present invention provides nanoparticle coatings, medical implants having nanoparticle coatings, and methods for their manufacture and use. In a broad aspect, the various embodiments of the nanoparticle coatings of the present invention comprise nanopulverized drug compounds. In a broad aspect, the nanoparticulate compounds comprise particles less than 500 nm in size. As those skilled in the art will appreciate, the smaller size of the compounds improves the solubility of these compounds as well as provides various benefits disclosed herein. For example, prolonged release of the drug compounds can be achieved as a result of the increased surface area of these compounds from nanopulverization. Furthermore, the smaller size of the drug compounds can improve the distribution of the compounds on the surface of a medical device.

According to one exemplary embodiment, the nanoparticle coatings comprise one or more drugs. The term “drug” as used herein means any compound intended for use in animals having a desired effect. Non-limiting examples include anticoagulants, such as an RGD (ARG-GLY-ASP) peptide-containing compound, heparin, antithrombin compounds, platelet receptor antagonists, anti-thrombin antibodies, anti-platelet receptor antibodies, aspirin, protaglandin inhibitors, platelet inhibitors, or tick anti-platelet peptide. Other classes of drugs includes vascular cell antiproliferative agents, such as a growth factor inhibitor, growth factor receptor antagonists, transcriptional repressor or translational repressor, antisense DNA, antisense RNA, replication inhibitor, inhibitory antibodies, antibodies directed against growth factors, cytotoxic agents, cytoskeleton inhibitors (eg: epothilone and taxol), peroxisome proliferator-activated receptor gamma (PPARγ) agonists, molecular chaperone inhibitors and bifunctional molecules. The drug can also include cholesterol-lowering agents, vasodilating agents, and agents which interfere with endogenous vasoactive mechanisms. Other examples of drugs can include anti-inflammatory agents, anti-platelet or fibrinolytic agents, anti-neoplastic agents, anti-allergic agents, anti-rejection agents, metaloprotease inhibitors, anti-microbial or anti-bacterial or anti-viral agents, hormones, vasoactive substances, anti-invasive factors, anti-cancer drugs, antibodies and lymphokines, anti-angiogenic agents, radioactive agents and gene therapy drugs, among others.

Specific non-limiting examples of drugs that fall under one or more of the above categories include paclitaxel, docetaxel and derivatives, epothilones, nitric oxide releasing agents, heparin, aspirin, coumadin, D-phenylalanyl-prolyl-arginine chloromethylketone (PPACK), hirudin, polypeptide from angiostatin and endostatin, benzoquinone ansamycins including geldanamycin, herbimycin and macbecin, methotrexate, 5-fluorouracil, estradiol, P-selectin Glycoprotein ligand-1 chimera, abciximab, exochelin, eleutherobin and sarcodictyin, fludarabine, sirolimus, rapamycin, ABT-578 (ABT-578 is a novel tetrazole-containing macrolide antibiotic. See U.S. Pat. No. 6,015,815 for non-limiting examples), certican, Sulindac, tranilast, thiazolidinediones including rosiglitazone, troglitazone, pioglitazone, darglitazone and englitazone, tetracyclines, VEGF, transforming growth factor (TGF)-beta, insulin-like growth factor (IGF), platelet derived growth factor (PDGF), fibroblast growth factor (FGF), RGD peptide, estrogens including 17 beta-estradiol and beta or gamma ray emitter (radioactive) agents.

In yet another embodiment, the antiproliferative compounds can include, but are not limited to, cytoskeletal inhibitors including, but not limited to, paclitaxel, cytochalasin, or analgoues thereof. In another embodiment, the antiproliferative compounds can include, but are not limited to, transforming oligonucleotides, and antisense oligonucleotides such as, but not limited to, c-myc and c-myb. As those skilled in the art will appreciate, it is contemplated that the drug Compounds of the present invention encompasses any antiproliferative or anti-restenotic compounds known or developed in the art.

In these exemplary embodiments of the present invention, the drug compounds can be nanopulverized by processes known or developed in the art. According to one method of the present invention, the drug compounds are nanopulverized by a mechanical process. For example the compounds can be nanopulverized into a fine powder by using techniques such as, but not limited to, a high-speed ball mill. Exemplary milling, precipitation, and homogenization methods of making nanoparticulate compositions are described in U.S. Pat. No. 5,145,684 (specifically examples 1-14 at columns 8-15), U.S. Pat. No. 5,518,187 (see columns 4-6, examples 1-6), U.S. Pat. No. 5,718,388 (see column 2 line 58 - (column 5 lines 36), U.S. Pat. No. 5,862,999 (see examples 1 and 2 at column 5 lines 35-column 6, line 17), U.S. Pat. No. 5,510,118 (see column 7 at line 38 through column 9 line 48), U.S. Pat. No. 5,766,635 (See column 3 at line 23 through column 4 line 22), and U.S. Pat. No. 6,428,814 (See Section D at column 19 line 40 to column 21 at line 10). The specific sections from the cited U.S. Patents describing milling, homogenization and nano-composition processing are herein specifically incorporated by reference.

For example, and not intended as a limitation, one exemplary nanopulverization, or milling process suitable for use with the present invention may include a dry process such as a dry milling process, or a wet process including wet-grinding. In one embodiment of the present invention this invention is practiced in accordance with the teaching of U.S. Pat. No. 5,145,684 (specifically examples 1-14 at columns 8-15). Thus, the wet grinding process can be practiced in conjunction with a liquid dispersion medium and surface modifier. Useful liquid dispersion media include water, aqueous salt solutions, ethanol, butanol, hexane, glycol and the like. The surface modifier can be selected from known organic and inorganic pharmaceutical excipients such as polyvinyl pyrrolidone (PVP).

In another embodiment the therapeutic agent can be prepared in nanoparticulate particle sizes less than about 500 nm. In other embodiments of the present invention particles having an average particle size of less than 100 nm have been prepared. Moreover, the present inventors have learned that fine nano-particulates as small as 10 nm may be prepared free of unacceptable contamination. Grinding can take place in any suitable grinding mill. Suitable mills include an airjet mill, an attritor mill, a vibratory mill, a sand mill and a bead mill. A high energy media mill is preferred especially when the grinding media is hard. The mill can contain a rotating shaft. This invention can also be practiced in conjunction with high speed dispersers such as a Cowles disperser, rotor-stator mixers, or other conventional mixers which can deliver high fluid velocity and high shear.

The preferred proportions of the grinding media, the therapeutic and/or diagnostic agent, the optional liquid dispersion medium, and surface modifier present in the grinding vessel can vary within wide limits and depends, for example, upon the particular therapeutic or diagnostic agent selected, the size and density of the grinding media, the type of mill selected, etc. Grinding media concentrations can range from about 10-95%, preferably 20-90% by volume depending on the application and can be optimized based on the above factors, milling performance requirements, and the flow characteristics of the combined grinding media and agent dispersion.

The attrition time can vary widely and depends primarily upon the particular therapeutic or diagnostic agent, mechanical means and residence conditions selected, the initial and desired final particle size and so forth. Residence times of less than about 8 hours are generally required using high energy dispersers and/or media mills. The process can be carried out within a wide range of temperatures and pressures. The process preferably is carried out at a temperature below that which can cause the agent to degrade. For many agents, ambient temperatures are appropriate. Temperatures of less than about 30° C. to 40° C. are typically preferred. Temperature control using jacketing or immersion of the milling chamber in ice water are contemplated. Processing pressures from about 1 psi (0.07 kg/cm²) up to about 50 psi (3.5 kg/cm²) are contemplated. Processing pressures from about 10 psi (0.7 kg/cm²) to about 20 psi (1.4 kg/cm²) are typical.

The therapeutic or diagnostic agent and the grinding media are continuously removed from the milling chamber. Thereafter, the grinding media is separated from the milled particulate agent (in either a dry or liquid dispersion form) using conventional separation techniques, in a secondary process such as by simple filtration, sieving through a mesh filter or screen, and the like. Other separation techniques such as centrifugation may also be employed. The present invention can be practiced with a wide variety of therapeutic and diagnostic agents. In the case of dry milling, the drug substances and imaging agents must be capable of being formed into solid particles. In the case of wet milling, the drug substances and imaging agents must be poorly soluble and dispersible in at least one liquid medium. By “poorly soluble”, it is meant that the therapeutic or diagnostic agent has a solubility in the liquid dispersion medium of less than about 10 mg/ml, and preferably of less than about 0.1 mg/mi. Additionally, the therapeutic compositions of the present invention any be milled into nano-particulars in any liquid medium in which they are poorly soluble.

According to one embodiment of the present invention, the nanopulverized pounds have substantially the same particle size. Alternatively, in another exemplary embodiment, the nanoparticle coatings of the present invention comprise nanopulverized drug compounds having a broad spectrum of particle sizes. For example, the nanoparticle coatings of the present invention can comprise drug compounds 10 nm to approximately 500 nm. As a result of the various compound sizes, controlled elution of these compounds can be achieved. That is, smaller nanoparticulate compounds will dissolve and released from the coating sooner than larger nanoparticulate compounds. As those skilled in the art will appreciate, by varying the ratio of differently sized nanoparticulate compounds, one can control the release rate of these compounds. For example, a nanoparticle coating comprising a greater number of 500 nm particles as compared to 10 nm particles can have prolonged drug release as it will take longer for the 500 nm particles to be broken down and then released from the coating. In contrast, the 10 nm particles do not need to be broken down into smaller particle sizes before they are released from the coating.

Another aspect of the present invention is directed to medical implants having drug compounds applied thereon. These medical implants include, but not limited to, stents, catheters, micro-particles, probes, vascular grafts, access devices, in-dwelling access ports, valves, plates, barriers, supports, shunts, discs, joints, as well as virtually any device intended for temporary or permanent implantation including implants that are bioresorbed. According to one embodiment, the nanoparticle compounds of the present invention are directly applied to at least one surface of a medical implant. In yet another embodiment of the present invention, the nanoparticle compounds are suspended, or blended, within a polymer. The polymer is then applied to at least one surface of the medical implant by techniques known or developed in the art.

Coating methods can vary depending on the surface type to be coated Exemplary coating methods include, but are not limited to, spraying, dipping, brushing, vacuum-deposition, and others. Moreover, the controlled release coatings of the present invention may be used with a cap coat. A cap coat as used here refers to the outermost coating layer applied over another coating. For examples, and not intended as a limitation: a metal stent has a parylene primer coat applied to its bare metal surface. Over the primer coating comprising a nanoparticle compound suspended or blended within a polymer is applied. Over the nanoparticle-containing polymer coating a polymer cap coat may be applied. The cap coat may optionally serve as a diffusion barrier to further control the drug release, or provide a separate drug. Alternatively the cap coat may be merely a biocompatible polymer applied to the surface of the sent to protect the stent and have no effect on elusion rates.

Exemplary bioabsorbable polymers that can be utilized to suspend nanoparticulate drug include, but are not limited to, either synthetic or natural bioabsorbable polymers. Synthetic bioabsorbable polymeric include, but are not limited to, poly (L-lactic acid), polycaprolactone, poly(lactide-co-glycolide), poly(ethylene-vinyl acetate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) such as PEO/PLA, polyalkylene oxalates, and polyphosphazenes. According to another exemplary embodiment of the present invention, the polymeric materials can be natural bioabsorbable polymers such as, but not limited to, fibrin, fibrinogen, cellulose, starch, collagen, and hyaluronic acid.

When a bioabsorbable polymer is used the nanoparticulate drug is dispersed within polymer, either evenly or in layers. The nanoparticulate-containing bioabsorbable polymer is then applied to the medical device surface in one, or a plurality of layers. The nanoparticulate compound can be evenly milled to a single particle size, or particles of various sizes can be provided. The rate of drug elution is then controlled by the rate of polymer absorption and particle size. Smaller nanoparticulate drug particles dissolve more quickly upon exposure to the physiological environment. Thus, a coating having a majority of 10 nm sized particles will tend to release drug into the body at rates much more quickly than coating having larger nanoparticulate drug particles. Moreover, the more rapidly the coating is absorbed, the more rapidly the drug, regardless of particle size will be releases.

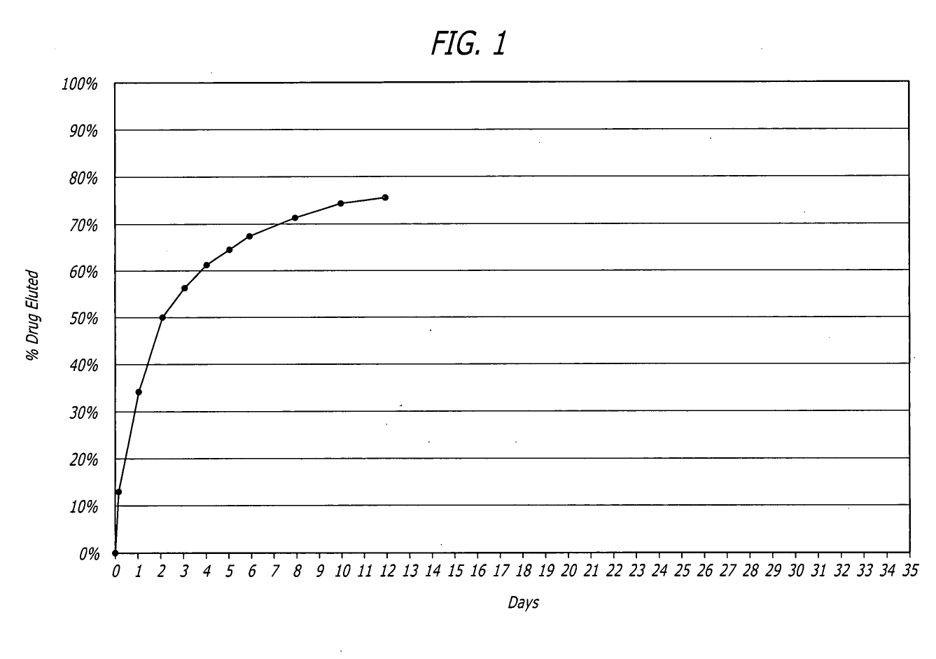

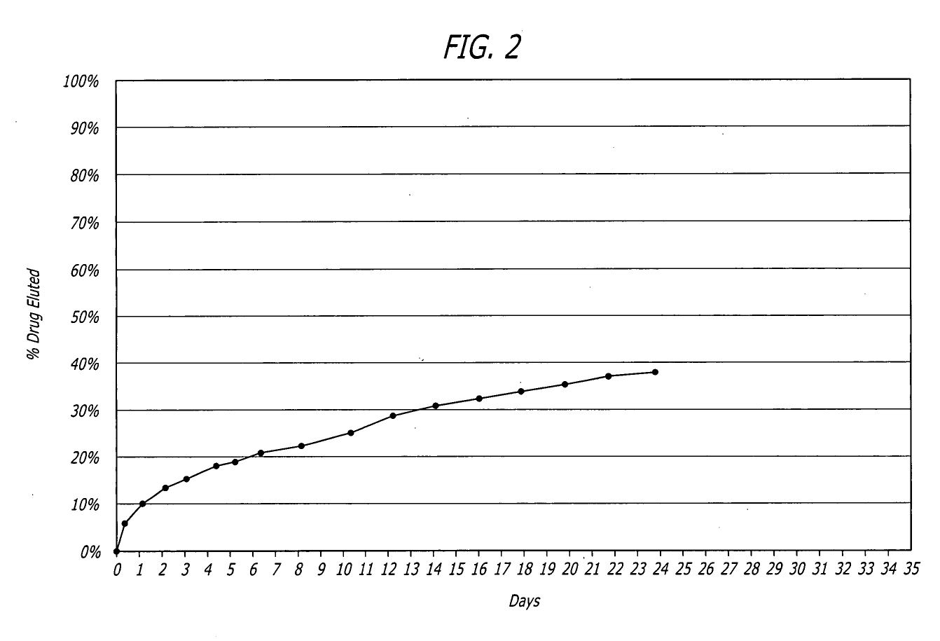

Therefore, when making the nanoparticulate coating systems of the present invention, polymer solubility, drug solubility and nanoparticulate drug size can be matched in ways that permit virtually any conceivable release rate or release kinetics. Generally speaking drug delivery rates have two primary kinetic profiles. Drugs that reach the blood stream or tissue immediately after administration follow first-order kinetics. FIG. 1 graphically depicts idealized first-order kinetics. First-order drug release kinetics provide an immediate surge in blood or local tissue drug levels (peak levels) followed by a gradual decline (trough levels). In most cases therapeutic levels are only maintained for a few hours. Drugs released slowly over a sustained time where blood or tissue concentrations remains steady follow zero-order kinetics. FIG. 2 graphically depicts idealized zero-order kinetics. Depending on the method of drug delivery and tissue/blood clearance rates, zero-order kinetics result in sustained therapeutic levels for prolonged periods. Drug-release profiles can be modified to meet specific applications. Generally, most controlled release compositions are designed to provide near zero-order kinetics. However, there may be applications where an initial burst, or loading dose, of drug is desired (first-order kinetics) followed by a more gradual sustained drug release (near zero-order kinetics). The present invention provides methods for designing nanoparticulate-drug-containing polymeric compositions having drug-release profiles that follow first-order kinetics, zero-order kinetics and first and zero-order kinetic combinations. Thus, among other qualities, the present invention provides polymeric controlled release coatings optimized for any application, specifically coatings for vascular implants.

For some applications polymer stability is a desirable quality. Thus non-bioresorbable polymers may be preferable. Thus in another embodiment of the present invention the nanoparticulate drug can be dispersed throughout non-bioabsorbable polymer or polymer blends. In this embodiment the polymer acts as a diffusion barrier and that participates in controlling the rate of drug diffusion from the medical device. Unlike coatings where in the drug is imbibed or dissolved in the coating polymers, the coating of the present invention obtain a higher level of control over drug delivery rates. For example, and not intended as a limitation, a therapeutic compound such as an anti-proliferative can be milled to form a nanoparticulate drug. The nanoparticulate drug can be uniformally sized or vary in size from 10 nm to 1000 nm. The nanoparticulate drug is then dispersed into a non-bioabsorbable polymer or polymer blend and then applied to the medical device surface using methods previously described. Suitable polymers include, but are not limited to polyesters, polyureas, polyurethanes, acrylates, acetates and the like. In one embodiment the polymer is a terpolymer-co-polymer blend that has been specifically compatiblized to match as closely as possible polymer-drug solubility factors.

The nanoparticulate-drug-containing non-bioresorbable polymers can be applied to the surface of the medical device in a single homogenous coating, or layered. Moreover, a plurality of layers can be applied each having the same or different nano-particulate drug dispersed therein. In another embodiment the nano-particluate drug can vary in size between layers, or be of a consistent size. Furthermore, different polymers can be used to form the various layers, each having the same or a different nanoparticulate drug dispersed therein. Again, the nano-particulate drug can vary in size or be consistent in size depending on the drug release rate and kinetics desired. The variations are myriad. The following exemplary embodiments provide teachings using non-resorbable polymers and are not intended as limitations on the inventive concepts discussed above. Rather, they serve to enable one having ordinary skill in the art how to control drug release rates and kinetics by matching suitable polymers with size of the drug nano-particle, nano-particle size distribution, the solubility of the drug in the polymer, the polymer stability (non-bioresorbable versus biorsorbable), coating thickness, the number of and the presence or absence of polymer layers, polymer primer coatings and/or cap coats.

Controlling the drug-release rate from medical device surfaces is challenging. This challenge is even greater when the device is a vascular stent. Stents are deployed in a physiological and anatomical environment that exposes the device and its coatings to the physical forces associated with blood circulation. Blood circulates throughout the body in a closed system of arteries and veins collectively referred to as blood vessels. The physical forces associated with blood circulation are referred to as hemodynamic forces and include the mechanical and hydrodynamic forces. For example, blood flow in and around vessel obstructions, including medical implants can result in turbulent blood flow. Turbulent blood flow can cause shear forces which have an eroding effect on exposed surfaces. Furthermore, there are mechanical forces associated with blood circulation such as the expansion and contraction of blood vessels and adjacent muscles and organs. These mechanical forces may cause vascular implants to bend, twist and strain and may also induce friction at the interface between the blood vessel lumen wall and vascular implant's surface. Finally, significant strain is placed on the stent body and coating during deployment. Stents are generally compressed on the distal end of the catheter and then expanded at the deployment site. The combination of pre-deployment compression and deployment expansion causes significant mechanical stress to the stent and any associated coating.

The present invention is directed at optimized drug releasing medical device coatings suitable for use in hemodynamic environments. The coatings of the present invention are composed of polymers having at least one nano-particularized bioactive agent dispersed therein. The polymeric compostions of the present invention have been specifically formulated to provide medical device coatings that tenaciously adhere to medical device surfaces (do not delaminate), flex without fracturing (ductile), resist erosion (durable), are biocompatible and release a wide variety of drugs at controlled rates.

Polymers have been used as medical device coatings for decades to enhanced biocompatibility and erosion resistance. Moreover, in certain applications polymer coatings may also provide electrical insulation. It is also well known in the art that polymers can act as reservoirs and/or diffusion barriers to control biological agent elution rates.

Recently, coatings have been applied to implantable medical devices such vascular stents, vascular stent grafts, urethral stents, bile duct stents, catheters, inflation catheters, injection catheters, guide wires, pace maker leads, ventricular assist devices, and prosthetic heart valves. Devices such as these are generally subjected to flexion strain and stress during implantation, application or both. Providing flexible medical devices such as stents with stable biocompatible polymer coatings is especially difficult.

There are two basic molecular morphologies that define a polymer's tertiary solid-state structure. Polymers may be either semi-crystalline or amorphous depending on the nature of the polymer subunit. Semi-crystalline polymers are ridged and brittle at any temperature below their melting point and are generally not suitable for coating flexible medical devices such as stents. In addition, drugs or bioactive agents can not stay in the polymer crystal region, therefore, the drugs or bioactive agents loading is limited. Amorphous polymers, on other hand, can be either rigid or elasticity/ductile depending on its glass transition point. The glass transition point of an amorphous polymer is the temperature above which the amorphous polymer is elastic/ductile and flexible. For stent application it is desirable that the Tg be below body temperature. Many polymeric compositions have glass transition points substantially above body temperature and are thus in the glassy or rigid state when the device is deployed and remains so once the device is implanted. Polymers in the “glassy” state are non- elastic/ductile and prone to cracking, fracturing and delaminating when the stent is flexed. Polymer coatings susceptible to fracture and delaminating are especially undesirable when used on stents. Small polymer particles that separate from a delaminated or fractured stent coating may be carried by the blood flow downstream where they can lodge in capillaries and obstruct blood flow to critical regions of the heart. Therefore stents and other flexible medical devices should have polymer coatings that are elastic/ductile and adhere to the device surface well. Generally, this requires that coating polymers be amorphous and have glass transition points (Tgs) below body temperature.

However, polymers having extremely low Tgs are undesirable when used to coat devices that are subjected to continual hemodynamic forces. As general rule, the lower the Tg the more rubbery a polymer becomes. More rubbery polymers can be tacky and less durable and are more likely to break down when exposed to hemodynamic induced stress and wear than less rubbery ones. This is partially due to the fact that the more rubbery polymers have higher coefficient of frictions and possess less structural integrity. Therefore, polymers having extremely low Tgs should not be the dominant polymer in polymer blends or copolymer compositions when designing coating polymers intend for stents and other vascular implants.

In addition to the aforementioned structural and drug releasing profile considerations, polymers used as stent coatings must also be biocompatible. Biocompatibility encompasses numerous factors that have been briefly defined in the preceding “Definition of Terms” section. The need for a polymer to be biocompatible significantly limits the number of available options for the material scientist. Moreover, these options are further limited when the polymer coating is used on a device that is continuously exposed to hemodynamic forces. For example, stent coatings must remain non-thrombogenic, non-inflammatory and structurally stable for prolong time periods.

There are generally two large, and to some extent overlapping, categories of biocompatible polymers suitable as medical device coatings: bioerodable (including bioabsorbable polymers) and non-bioerodable polymers. The present invention's methods are equally applicable to bioreodable and non-bioerodable polymer coatings. The remaining discussion and exemplary embodiments will be directed at non-bioerodable polymers.

Non-erodable polymers can be hydrophilic, hydrophobic or amphiphilic depending on the polarity of the monomers used and the ratio of hydrophobic to hydrophilic monomers. Hydrophilic polymers are polar molecules that are miscible with polar solvents and are generally lubricious while contacting body fluids. Hydrophilic polymers are often used in biomedical applications to produce lubricious hydrogels.

Hydrogels are an exceptionally diverse group of materials. Virtually all hydrophilic polymers can be crosslinked to produce hydrogels, whether the polymer is of biological origin, semi-synthetic, or wholly synthetic. Hydrogels properties such as equilibrium swelling degree, sorption kinetics, solute permeability, and their in vivo performance characteristics possess properties that make them suitable for drug delivery applications. The equilibrium swelling degree or sorption capacity (swollen volume/dry volume) is the single most important property of a hydrogel and directly influences the other properties. Unfortunately, the mechanical strength of a gel declines in rough proportion to the swelling degree, although strength is usually of lesser concern for drug delivery than the other four properties. However, when mechanical strength is important for a hydrogel, it can be bonded onto a support made of plastic, ceramic or metal. The composite system then gains the mechanical strength of the substrate along with the useful drug delivery properties of the hydrogel.

These properties, either singly or in combination, have led to widespread interest in the use of hydrogels for drug delivery. These materials can be used to protect labile drugs from denaturants, control the release rate of the therapeutic agent, or help target release to a chosen site within the body. They can be made as oral dosage forms, suppositories, injectable micro/nanoparticles, or implants at any site within the body, even when blood contact is required. (For examples see: S. H. Gehrke “Synthesis and Properties of Hydrogels for Used for Drug Delivery,” Transport in Pharmaceutical Sciences, G. Amidon and P. I. Lee, Eds., Marcel Dekker, New York, in press (1999). The entire contents of which are herein incorporated by reference). However, hydrogel polymers can be unstable in a hemodynamic environment and lack physical integrity because their high water content. Classically, many hydrophobic drugs do not disperse well in hydrogels and therefore hydrogels are not considered ideal drug delivery platforms for some hydrophobic bioactive agents. However, when the drug is nano-particularized in accordance with the teachings of the present invention it can be stably incorporated into the hydrogel matrix and release rates are thus more easily controlled.

However, the swellability of hydrogels and other hydrophilic polymers can be a relevant consideration when making the compatibilized drug-releasing coatings of the present invention. Swellabilities of polymers in water can be easily determined. It should be understood, however, that the swellability results from incorporation of water and not from an elevation in temperature. As water is incorporated into a swelling polymer the free volume increases. As a result, the rate at which a drug will diffuse from a polymer matrix increases and the polymer becomes increasingly elastic/ductile. Therefore, by selecting relatively low and high swell polymers that are miscible, the dissolution rate and can be adjusted (the effect of increasing free volume on elasticity/ductility will be discussed in more detail below). This is particularly true for polymer matrices incorporating high molecular weight bioactive agents or nano-particles larger than 500 nm and can be a major contributing factor to elution rates for matrix systems as will be discussed more fully below.

Preferably, a polymer having a relatively high swellability is combined with a polymer having a relatively low swellability. For example, a miscible polymer blend for an active agent having a molecular weight of greater than 1200 g/mol includes polyvinyl pyrollidone-vinyl acetate copolymer, which has a swellability of greater than 100% (i.e., it is water soluble), and poly(ether urethane), which has a swellability of 60%. By combining such high and low swell polymers, the active agent delivery system can be tuned for the desired dissolution time of the active agent.

Swellabilities of the miscible polymer blends are also used as a factor in determining the combinations of polymers for a particular active agent. For delivery systems in which the active agent has a molecular weight of greater than 1200 g/mol, whether it is hydrophilic or hydrophobic, polymers are selected such that the swellability of the blend is greater than 10% by volume. The swellability of the blend is evaluated without the active agent incorporated therein.

Hydrophobic polymers such as polytetrafluoroethylene (PTFE AKA Teflon®) do not swell but can also be biocompatible. Teflon® has an extremely low coefficient of friction and is one of the most widely used hydrophobic biocompatible polymers. However, PTFE's slipperiness makes it difficult to handle and manipulate. Moreover, PTFE is a stiff chemical inert polymer and bonds poorly to surfaces. Furthermore, PTFE's extremely hydrophobic nature significantly limits its chemical compatibility with many bioactive agents. Recently, nanoporous PTFE has been developed that can be used as a barrier coating, or cap coat, that mediates bioactive agent release from an underlying drug reservoir (Advanced Surface Engineering, Inc. Eldersburg, Md.). However, nanoporous PTFE coatings are expensive and the application process is not compatible with all medical device surfaces and drug categories. Consequently, the usefulness of PTFE as a medical device coating is limited. There are many other biocompatible hydrophobic polymers; however, many of these have a high coefficient of frictions which is undesirable in a hemodynamic environment. Moreover, many hydrophilic drugs do not disperse well in hydrophobic polymer and therefore are not suitable drug delivery platforms for many hydrophilic bioactive agents. However, when the drug is nano-particularized in accordance with the teachings of the present invention it can be stably incorporated into PTFE and release rates are thus more easily controlled.

Therefore, there are four specific attributes that the stent coating polymers made in accordance with the teachings of the present invention should possess. The polymer compositions of the present invention should be biocompatible, durable, elastic/ductile and possess a predetermined drug release profile. Prior to the present invention polymer coating design was largely a matter of trial and error. Previously, material scientists based their polymer coating compositions on best guesses and previous experience. Slight modifications were made randomly and the resulting polymer compositions were tested in vitro and in vivo. Unsuccessful polymers were eliminated from further consideration and the successful polymer ultimately selected from myriad potential candidates.

The present invention provides methods for reproducibly balancing the four critical parameters of biocompatibility, durability, elasticity/ductility and drug release profile when designing controlled release polymer coatings. Moreover, the present invention provides therapeutic compositions that are nano-pulverized and incorporated into the coating polymer. This added dimension of controlling drug particle size combined with polymer biocompatibility, durability and elasticity/ductility provides material scientists more control over drug elusion rates and elusion kinetics than previous possible.

One of the most fundamental physical chemical properties that must be considered when selecting polymers for use as controlled release coatings is the polymer's solubility parameters. The present inventors have developed a novel method of using solubility parameters for designing polymeric compositions useful as controlled release coatings for devices deployed in hemodynamic environments. At its most basic level, the present invention employs principles of polymer physical chemistry to match polymeric compositions with drugs so that the resulting controlled release coatings have both optimum physical attributes and drug release kinetic profiles.

As used hereinafter the compositions of the present invention will be referred to as “controlled release coatings.” This terms shall referred to a polymeric composition that has optimum physical characteristics such including biocompatibility, durability, elasticity/ductility in addition to a predetermined optimum drug releasing kinetic profile made possible in part due to drug nano-pulverization and particle sizing as taught in accordance with the present invention.

Polymer solubility parameters as a function of a polymer's cohesive properties were known to be a direct expression of the polymer's behavior in aqueous and organic solvents as early as 1916. However, it was not until 1949 that Hildebrand proposed the term solubility parameter and assigned the symbol “δ” to represent a polymer's behavior in specific solvents; as previously discussed, “δ” will be expressed in J^{1/2}/cm^{3/2}. However, Hildebrand had only considered dispersive forces between various structural units when determining solvent/polymer solubility parameters. Later, Hansen et al. established that the interaction between polar groups and hydrogen bonding contributed significantly to the total cohesive energy, and thus the solubility behavior of many liquids and amorphous polymers. Therefore, Hansen defined a polymer's total solubility (δ_{T}) as the interaction between three distinct values: dispersion force (δ_{D}), polar force (δ_{P}), and hydrogen bonding force (δ_{H}) (see van Krevelen at pages 189-226). As used herein δ_{T} will be used to refer to the final solubility parameter of a controlled release coating made in accordance with the teachings of the present invention. As will be evident from the teachings that follow, a controlled release coating may be a terpolymer, or a blend of copolymers and/or copolymers and homopolymers.

The present inventors have used the Hansen solubility parameters to optimize controlled release coating compositions for stents. The present inventors have determined that the optimum drug release kinetic profile occurs when the polymer's solubility parameter closely matches the drug's. However, merely matching candidate drug's solubility with polymer's δ_{T} does not always result in a functional controlled release coating. As discussed extensively above there are three additional criteria that a successful controlled release coating must meet. The present inventors have determined that various homoploymers, copolymer and combinations thereof, can be designed by balancing the Hansen solubility parameters of the polymer subunits and/or individual polymers in a blend.

Generally speaking for an individual homopolymer's δ equals Σδ_{D}+δ_{P}+δ_{H}. In a copolymer the combined δ_{S}, or total δ (δ_{T}), equals ΣXδ₁+Xδ₂+Xδ₃+Xδ₄ . . . where X equals the percentage of each polymer subunit (T1, T2, T3 etc) in the total polymer. Likewise, for a polymer blend the combined δ_{T} equals ΣXδ_{T1}+Xδ_{T2}+Xδ_{T 3}+Xδ_{T4} . . . where X equal percentage of each individual polymer in the blend (T1, T2, T3 etc). Therefore, the present inventors determined that the δ_{T} can be adjusted to match the δ for any given drug.

However, the present inventors have also determined that a copolymer's biocompatibility, elasticity/ductility and durability can be optimized by altering the ratio of polymeric subunits that favor one property over another. For example, ductility and durability are roughly a function of the polymer's Tg. The lower the Tg, the more ductile the polymer becomes. However, below a certain point the polymer becomes too rubbery and its durability is adversely effected. Moreover, extremely rubbery polymers possess greater first-order kinetics than near zero-order kinetics, consequently, extremely low Tgs are to be avoided.

The present inventors have developed a system for controlled release coating design that is conceptually similar to how the individual Hansen solubility parameters affect a polymer's δ. For example, hydrogen bonding capacity contributes more significantly to a controlled release coating's biocompatibility than other factors. Therefore, polymers having high hydrogen bonding potential such as poly(N-vinyl pyrrolidone) increase biocompatibility.

Elasticity/ductility increases as the polymer's Tg decreases. Tg in turn decreases as the polymer's free volume increases. Free volume corresponds to the unoccupied regions accessible to segmental motions. Free volume in turn is affected by several factors including swellability, the number and size of pendent groups present on polymer subunits and the extent and degree of cross linking. The affects of free volume on Tg are best appreciated with reference to examples. Compare the effects on Tg caused by adding two different alkyl ester monomers to a terpolymer. For this example assume that the terpolymer is composed of 30% vinyl acetate, 40% Y-methacrylate and 30% N-vinyl pyrrolidone. If Y equals hexyl, the resulting terpolymer has a calculated Tg of 21° C. and a calculated δ of approximately 21. However, if lauryl methacrylate is substituted for hexyl-methacrylate the polymers' Tg to -11 but δ remains approximately 21.

The preceding example demonstrates the effect that pendent chain group size has on Tg. Note that the larger pendent group on the lauryl methacrylate monomer dramatically decreases Tg as compared to hexyl-methacrylate while having no effect on δ. However, as previously stated, even though δ remains the same, the release kinetics of the terpolymer using lauryl methacrylate in place of hexyl-methacrylate are not optimum. The lauryl methacrylate-containing polymer exhibited near first-order kinetics when tested with drug solubilized in the polymer mix. Therefore, the present inventors have discovered that the pendent group size on hydrophobic polymers such as alkyl esters can affect polymer dispersive forces (δ_{D}) and which in turn can affect release kinetics and Tg. However, Tg can also be affected by monomer polarity (δ_{P}). In one embodiment of the present invention vinyl acetate is used to modulate polymer polarity. Vinyl acetates have polar groups that increase the intermolecular forces and decrease free volume. As free volume decreases, Tg increases. Importantly, controlling drug partilce size can minimize free volume considerations. Thus, using large nano-particles to gain better control over release rates may optimize polymer formulations that possess undesirable release kinetics as a result of having excessive polymer free volume. Consequently, the present invention makes previously unacceptable polymer compositions useful as drug releasing coatings.

In another embodiment compatible polymer blends are made using the teachings of the present invention. As used herein compatible polymer blends shall mean two or more chemically distinct polymers, including homopolymers and copolymers that form a stable mixture that does not separate on standing or during prolonged use and possess the other desired physical and chemicals properties discussed herein. Methods for compatibilizing two or more polymers with at least one bioactive agent are provided accordingly. A first polymer composition known to have certain desirable properties such as biocompatibility and elasticity/ductility is selected. However, the Tg of the first polymer may be below the desired range and thus have poor controlled release properties (for example it may have a first-order kinetic profile). Moreover, the first polymer may not have a δ compatible with the bioactive agent. Consequently, a second polymer composition having solubility parameters and Tg that balance the first polymer's Tg and δ can be blended with the first polymer composition to create an optimum controlled release coating.

For example, and not intended as a limitation, polyethylene-co-vinyl acetate (PEVAc) copolymers are durable, elastic/ductile and exhibit good adhesion to metals. However, PEVAc's low Tg renders the polymer tacky and prone to first-order drug release kinetics. Therefore, it may be desirable to create polymer blends using PEVAc as the first polymer composition and a second polymer that is compatible with PEVAc. Moreover, the second polymer component should have a Tg that compensates for PEVAc's low Tg and also possess δ values that provide a δ_{T} for the polymer blend that approximately matches the bioactive agent's δ. However, in order to assure that the polymer blend that has a δ_{T} and Tg optimized for a controlled release coating the first and second polymer composition must be miscible. In this example, because PEAVc contains vinyl acetate the second polymer composition should possess vinyl acetate monomers to increase miscibility. This approach helps compatibilize the second polymer composition with PEVAc and helps provide a compatible polymer blend having an optimized drug release kinetic profile.

Therefore, optimized controlled release coatings require that both δ values and Tg be considered when selecting polymer subunits. The design of a controlled release coating made in accordance with the teachings of the present invention begins with selecting the drug or drug-combination to be delivered and the desired drug release kinetic profile. Next the drug's solubility parameter is determined and the general chemical make up of the molecule is considered, that is, is the drug polar or non-polar? Then, starting with the basic assumption that like-dissolves-like, polymer units having a δ_{P} approximately equal to the drug are selected. However, it is important to avoid selecting alkyl esters having extremely large pendent groups that may drop the controlled release coating's Tg below that optimum for the desired release kinetics. Polymer subunits are then selected having δ_{D}'S that will balance the adverse effects on Tg caused by polymers having an unfavorable δ_{P}. Finally, polymer subunits having δ_{H}'S necessary for good biocompatibility are added to the list of candidate polymeric subunits.

Adjustments to the theoretical polymer blends can be made by varying polymer subunit concentrations in accordance with the teachings of the present invention until a δ_{T} approximately equal to the drug's δ is achieved. If the Tg drops below an acceptable range for the drug release kinetics desired the δ_{P} and δ_{D} components can be adjusted, or slightly different polymeric subunits can be selected as necessary. Finally, once the desired Tg range is reached the final concentration of δ_{H} subunits can be adjusted to assure optimum biocompatibility. The final polymer, or polymer blend, will have a δ_{T} approximately equal to the drug's δ and a Tg below body temperature, but not so low as to adversely affect the drug release kinetic profile desired. In one embodiment of the present invention δ is between approximately 15 J^{1/2}/cm^{3/2} to 21 J^{1/2}/cm^{3/2} and Tg is between approximately 10° C. and 35° C.

Persons having ordinary skill in the art will realize that the above discussion is intended as a guide and that minor variations can be made to the order of polymer selection and target Tg and δ values without deviating from the sprit of the invention. Moreover it is with the scope of the present invention to add polymer modifiers including crosslinking reagents and polymer grafts to control swelling and enhance over all durability and drug release kinetics.

However, release rate is not entirely a function of drug-polymer compatibility. Coating configurations, polymer swellability and coating thickness also play roles. When the medical device of the present invention is used in the vasculature, the coating dimensions are generally measured in micrometers (μm). Coatings consistent with the teaching of the present invention may be a thin as 1 μm or a thick as 1000 μm. There are at least two distinct coating configurations within the scope of the present invention. In one embodiment of the present invention the drug-containing coating is applied directly to the device surface or onto a polymer primer coat such a parylene or a parylene derivative. Depending on the solubility rate and profile desired, the drug is either entirely soluble within the polymer matrix, or evenly dispersed throughout. The drug concentration present in the polymer matrix ranges from 0.1% by weight to 80% by weight. However, drug solubility as used herein does not imply that the drug is uniformly dispersed with in the polymer. Rather, it implies that the drug can diffuse through the polymer and into the surrounding tissues. Thus particle size can be used to provide a heterologous matrix where drug nano-particles are dispersed throughout the polymer. The partilce size thus controls that rate at which the drug disperses within the polymer and thew polymer's compatibility with thew drug, as determined by their relative solubility parameters helps control migration from the polymer coating and into the surrounding tissues.

In another embodiment of the present invention a drug-free polymer barrier, or cap, coat is applied over the drug-containing coating. The drug-containing coating serves as a drug reservoir. Generally, the concentration of drug present in the reservoir ranges from abort 0.1% by weight to as much as 100%. The barrier coating participates in the controlling drug release rates in at least three ways. In one embodiment the barrier coat has a solubility constant different from the underlying drug-containing coating. In this embodiment the drugs diffusivity through the barrier coat is regulated as a function of the barrier coating's solubility factors. The more miscible the drug is in the barrier coat, the quicker it will elute form the device surface and visa versa. This coating configuration is commonly referred to as a reservoir coating.

In another embodiment the barrier coat comprises a porous network where the coating acts as a molecular sieve. The larger the pores relative to the size of the drug, the faster the drug will elute. Moreover, intramolecular interactions will also determine the elution rates. The intramolecular interactions having the greatest net effect on drug elution include the relative hydrophobicity/hydrophilicity (δ_{H}) of the drug-polymer interaction. These factors have already been discussed above and apply to both the drug-containing coating as well as the barrier coating, the less intramolecular interaction between the drug and polymer barrier coat, the faster the drug will transit the porous network and enter the neighboring tissues. Persons having ordinary skill in the art of material science in combination with the teachings herein will readily understand that many variations on the cap coat and drug-eluting coatings can be made to tune the target diffusivity of the present invention.

Swellability is also an important factor. Polymer free volume increases proportionally to increases in swellability. Therefore, drug elution rate, as well as Tg increase with increasing swellability. As a result, for the purposes of the present invention the total swellability of the polymer blend used with bioactive agents having molecular weights less or than about 1200 g/mol and polymer blends having a δ_{T} greater than 25 J^{1/2}/cm^{3/2} should not exceed 10% by volume. Moreover, the total swellability should not exceed 10% by volume when the active agents have molecular weights greater than about 1200 g/mol and the polymer blend has a δ_{T} less than 25 J^{1/2}/cm^{3/2}. In both cases this remains true regardless of whether the bioactive agent is hydrophilic or hydrophobic.

Finally, returning to coating thickness, while thickness is generally a minor factor in determining overall drug-release rates and profile, it is never-the-less an additional factor that can be used to tune the coatings. Basically, if all other physical and chemical factors remain unchanged, the rate at which a given drug diffuses through a given coating is directly proportional to the coating thickness. That is, increasing the coating thickness increases the elution rate and visa versa.

We now turn to another factor that contributes to the compatiblized controlled release coatings of the present invention. As mentioned earlier, coating intended for medical devices deployed in a hemodynamic environment must possess excellent adhesive properties. That is, the coating must be stably linked to the medical device surface. Many different materials can be used to fabricate the implantable medical devices including stainless steel, nitinol, aluminum, chromium, titanium, ceramics, and a wide range of plastics and natural materials including collagen, fibrin and plant fibers. All of these materials, and others, may be used with the controlled release coatings made in accordance with the teachings of the present invention.

There are many theories that attempt to explain, or contribute to our understanding of how polymers adhere to surfaces. The most important forces include electrostatic and hydrogen bonding. However, other factors including wettability, absorption and resiliency also determine how well a polymer will adhere to different surfaces. Therefore, polymer base coats, or primers are often used in order to create a more uniform coating surface. In one embodiment of the present invention medical devices, specifically stents, are provided with polymer primer coats that provide inert adhesion layers for the controlled release coatings of the present invention. For example, and not intended as a limitation, parylene C is applied to the stent surface using vapor deposition techniques. Parylene is a hydrophobic, biocompatible, lubricious polymer that is transparent, flexible and meets USP class VI plastic requirements. Moreover, parylene is a gas-phase polymerized composition that completely forms to device surface topologies leaving a thin, pinhole-free base coat that is readily coated with other polymers. Parylene's hydrophobic nature can present challenges to coating scientists. However, when used in accordance with the teaching of the present invention, controlled release polymer compositions can be optimized to assure good long-term adhesion to the primer coat.

The controlled release coatings of the present invention can be applied to medical device surfaces, either primed or bare, in any manner known to those skilled in the art. Applications methods compatible with the present invention include, but are not limited to, spraying, dipping, brushing, vacuum-deposition, and others. Moreover, the controlled release coatings of the present invention may be used with a cap coat. A cap coat as used here refers to the outermost coating layer applied over another coating. For examples, and not intended as a limitation: a metal stent has a parylene primer coat applied to its bare metal surface. Over the primer coat a drug-releasing terpolymer coating or blend of homopolymer, copolymer and terpolymer coating is applied. Over the terpolymer a polymer cap coat is applied. The cap coat may optionally serve as a diffusion barrier to further control the drug release, or provide a separate drug. The cap coat may be merely a biocompatible polymer applied to the surface of the sent to protect the stent and have no effect on elusion rates.

The following non-limiting examples illustrate some of the various aspects of compositions and methods used to provide implantable medical devices with controlled release coatings. Various polymer compositions were prepared and analyzed in accordance with the teachings of the present invention. The present inventors have determined that the drug release rates and profiles are optimum if the polymer's total solubility parameter (δ_{T}) is approximately equal to a bioactive agent's solubility parameter (δ). For the purposes of the present invention the polymer's total solubility parameter (δ_{T}) is considered approximately equal to a bioactive agent's solubility parameter (δ) if their respective δ values fall with in plus or minus 10 J^{1/2}/cm^{3/2}, and/or the difference between at least one solubility parameter of each of the at least two polymers is no greater than about 5 J^{1/2}/cm^{3/2}.

Furthermore, in one embodiment of the present invention compatible polymer blends are made wherein the ratio of low Tg polymer to high Tg polymer is in the range of 20:80 to 80:20. In one particular embodiment the ratio of low Tg polymer to high Tg polymer is 50:50. In another embodiment the ration of low Tg polymer to high Tg polymer is 60:40. In another embodiment the ration of low Tg polymer to high Tg polymer is 70:30 In another embodiment the ration of low Tg polymer to high Tg polymer is 80:20. It is understood that these ratios and ranges are approximate and that the exact ratio of low Tg polymer to high Tg polymer is determined in accordance with the present teachings.

For exemplary purposes two anti-restenotic, bioactive compositions were used to test the controlled release kinetics of the present invention. The solubility parameter for each drug is 17.5 δ. The drugs were given the laboratory designators A-19 and A-20. Table I lists the copolymers used in the following exemplary embodiments. These polymers were prepared using methods known to those skilled in the art of polymer chemistry and as detailed in references such as, A. Rawe. Principles of Polymer Chemistry, Second Edition. 2000. Kluwer Academic/Plenum Publishers, New York. ISBN 0-306-46368-7; H. Allcock and F. Lampe. Contemporary Polymer Chemistry. 1981. Prentice-Hall, New Jersey. ISBN 0-13-170258-0. and A. Tonelli. Polymers from the Inside Out. 2001. Wiley-Interscience. ISBN 0-471-38138-1. All of these references are incorporated by reference herein; additional exemplary teaching are also provided.

The following abbreviations will be used in referring to the various exemplary polymer compositions: VAc=vinyl acetate monomer; BMA=butyl methacrylate monomer; HMA=hexyl methacrylate monomer; LMA=lauryl methacrylate monomer; NVP=N-vinyl pyrrolidone monomer and PEVC=(poly) ethylene-vinyl acetate copolymer.

Exemplary embodiments of non-bioabsorbable polymers suitable for use in conjunction with the nano-particles of the present invention include the following:

### General Method of the Two-Step Synthesis of Segmented N-Butyl Methacrylate and Vinyl Acetate Copolymers

One embodiment of the present invention is exhibited by a two-step synthesis of a copolymer with n-butyl methacrylate and vinyl acetate segments. In the first step of the synthesis, predetermined amounts of n-butyl methacrylate (BMA) and vinyl acetate (VAc) were mixed in a pre-dried glass reactor equipped for mechanical stirring while providing a nitrogen environment about the reactants. The mixture was then sparged with nitrogen for about five minutes. A requisite amount of azo-bis-butyronitrile (Azo) was added to the mixture. In most cases, isopropyl alcohol (IPA) sparged with nitrogen was also added to the mixture. The mixture was heated to the desired temperature under nitrogen and stirred for a certain period of time until the commencement of the second step.

In the second step of the synthesis, a second aliquot of the Azo free radical initiator and IPA were added prior to introduction of a second charge of monomer or comonomer. The monomer and comonomer were also sparged with nitrogen. The polymerization was continued at the desired temperature until monomer consumption practically ceased, maintaining agitation while possible.

At the conclusion of the second step, the heating was stopped and the product was mixed in the reactor with a suitable solvent such as acetone to facilitate the polymer purification by precipitation in a cold non-solvent such as water or methanol or a mixture thereof. The precipitated copolymer was then isolated by filtration and allowed to dry in a laminar flow hood under reduced pressure at room temperature until a constant dry weight was achieved. Further drying can be accomplished by heating under reduced pressure until a constant dry weight is achieved.

The following abbreviations will be used in referring to the various exemplary polymer compositions: VAc=vinyl acetate monomer; BMA=butyl methacrylate monomer; HMA=hexyl methacrylate monomer; LMA=lauryl methacrylate monomer; NVP=N-vinyl pyrrolidone monomer and PEVc=(poly) ethylene-vinyl acetate copolymer.

TABLE 1
Copolymer and Terpolymer Compositions

| | mole % of each | Polymer | | |
|---|---|---|---|---|
| Polymer Composition | monomer | ID | δ | Tg (° C.) |
| VAc:nBMA | 5/95 | A | 18.0 | 20.6 |
| VAc:nBMA | 10/90 | B | 18.1 | 21.0 |
| VAc:nBMA | 70/30 | C | 19.1 | 28 |
| VAc:nHMA:NVP | 7-30/40-75/19-30 | D | | |
| VAc:nHMA:NVP | 30/40/30 | D1 | 21.0 | 21.0 |
| VAc:nHMA:NVP | 20/60/20 | D2 | 17.8 | 12.2 |
| VAc:nHMA:NVP | 10/70/20 | D3 | 17.9 | 8.6 |
| VAc:nHMA:NVP | 9/71/20 | D4 | 17.9 | 8.2 |
| VAc:nHMA:NVP | 7/73/20 | D5 | 18.0 | 7.5 |
| VAc:nHMA:NVP | 7/74/19 | D6 | 18.0 | 7.0 |
| VAc:nLMA:NVP | 30/40/30 | E | 21.0 | −11 |

Table 2 represents the exemplary polymer blend prepared in accordance with the teachings of the preset invention and the resulting solubility (δ_{T})value for each polymer blend. The blends were prepared such that the resulting δ_{T} fell between 15 and 21 δ to be compatible with drugs' δ of 17.5.

TABLE 2
Exemplary Compatibilized Controlled Release Coatings

| | Percent | | |
|---|---|---|---|
| | Monomer | | |
| | Sub-unit | Polymer | |
| Compatibilized Polymer Blend | Component1 | Blend ID | δ_{T} |
| PEVAc:Polymer A | 50/50 | I | 17.7 |
| PEVAc:Polymer A | 60/40 | II | 17.8 |
| PEVAc:Polymer B | 50/50 | III | 17.8 |
| PEVAc:Polymer B | 40/60 | IV | 17.8 |
| PEVAc:Polymer B:Polymer C | 40/50/10 | V | 17.9 |
| PEVAc:Polymer B:Polymer C | 40/40/20 | VI | 18.0 |
| PEVAc:Polymer B:Polymer C | 50/41.7/8.3 | VII | 17.8 |
| PEVAc:Polymer B:Polymer C | 50/33.3/16.7 | VIII | 17.9 |
| PEVAc:Polymer B:Polymer C | 60/33.3 | IX | 17.8 |
| PEVAc:Polymer B:Polymer C | 60/26.7/13.3 | X | 17.8 |
| PEVAc:Polymer E | 20/80 | XI | 20.2 |
| Polymer B:Polymer D1 | 80/20 | XII | 18.0 |
| Polymer B:Polymer D1 | 70/30 | XIII | 18.0 |
| Polymer B:Polymer D1 | 60/40 | XIV | 18.0 |
| Polymer B:Polymer D1 | 50/50 | XV | 18.0 |
| Polymer B:Polymer D1 | 40/60 | XVI | 18.0 |

1The percent monomer sub-unit component is measured on a weight-percent basis.

### EXAMPLE 1 A

### General Method of the Two-Step Synthesis of Segmented n-Butyl Methacrylate and Vinyl Acetate Copolymers

One embodiment of the present invention is exhibited by a two-step synthesis of a copolymer with n-butyl methacrylate and vinyl acetate segments. In the first step of the synthesis, predetermined amounts of n-butyl methacrylate (BMA) and vinyl acetate (VAc) were mixed in a pre-dried glass reactor equipped for mechanical stirring while providing a nitrogen environment about the reactants. The mixture was then sparged with nitrogen for about five minutes. A requisite amount of azo-bis-butyronitrile (Azo) was added to the mixture. In most cases, isopropyl alcohol (IPA) sparged with nitrogen was also added to the mixture. The mixture was heated to the desired temperature under nitrogen and stirred for a certain period of time until the commencement of the second step.

In the second step of the synthesis, a second aliquot of the Azo free radical initiator and IPA were added prior to introduction of a second charge of monomer or comonomer. The monomer and comonomer were also sparged with nitrogen. The polymerization was continued at the desired temperature until monomer consumption practically ceased, maintaining agitation while possible.

At the conclusion of the second step, the heating was stopped and the product was mixed in the reactor with a suitable solvent such as acetone to facilitate the polymer purification by precipitation in a cold non-solvent such as water or methanol or a mixture thereof. The precipitated copolymer was then isolated by filtration and allowed to dry in a laminar flow hood under reduced pressure at room temperature until a constant dry weight was achieved. Further drying can be accomplished by heating under reduced pressure until a constant dry weight is achieved.

### EXAMPLE 1 B

### General Methods of Analysis

A set of general analysis methods was used to monitor and characterize the polymerization reactions. In-process monitoring of the polymerization reaction was achieved by the analysis of residual monomers and molecular weight build-up using gel permeation chromatography (GPC) with dichloromethane as a solvent.

The purified copolymer was characterized with infrared analysis using a film prepared from a chloroform solution. The composition of the purified copolymer was determined with nuclear magnetic resonance (NMR), using CDCL₃ as a solvent. Weight average molecular weight was measured using GPC with dichloromethane (DCM) or tetrahydrofuran (THF) as a solvent, and the inherent viscosity (I.V.) with chloroform.

### EXAMPLE 1 C

### General Method of Film Formation and Determination of Percent Elongation

Fracture strain characteristics of the polymeric material may be measured by forming the polymer into a sheet, and applying strain to a sample of the material, and determining when the sample breaks, thereby determining the fracture strain.

The dried polymer was compression-molded into a film about 0.1 mm thick using a heated laboratory Carver press. The temperature, pressure, and time used varied with the copolymer composition—typically above 50° C., 3,000 lbs, and 2 minutes, respectively. The pressed polymer was then quick-quenched to about 25° C. and removed. The molded film was cut into 13×40 mm pieces. The percent elongation was determined on a Mini-Bionix Universal Tester using a gauge length of 19 mm and strain rate of 0.5 mm/s.

### EXAMPLES 2A-2J

### Two-Step Synthesis of VAc and BMA Segmented Chain Copolymer With BMA in the Second Step

A segmented chain copolymer was synthesized in a two-step procedure using only BMA in the second step. Reaction charge and conditions for the preparation of ten copolymers are summarized in Table 3A and Table 3B. The copolymers were made using a (1) high VAc to BMA ratio and only BMA in the second step; (2) polymerization temperature of 55° C. to 70° C.; and (3) no or variable amounts of IPA.

TABLE 3A
Two-step Synthesis and Properties of Segmented VAc to BMA
Copolymers Using only BMA in the Second Step

| Example Number | 2A | 2B | 2C | 2D | 2E |
|---|---|---|---|---|---|
| Step 1 | | | | | |
| VAc/BMA (g/g) | 8/2 | 23/6 | 94/100 | 90/10 | 80/20 |
| Azo (mg) | 38 | 100 | 249 | 249 | 300 |
| IPA (ml) | 0 | 0 | 100 | 10 | 10 |
| R Time (hour)/Temp | 1/70 | 1/70 | 20/60 | 23/60 | 53/55 |
| (° C.) | | | | | |
| Step 2 | | | | | |
| BMA/VAc (g/g) | 8/0 | 22/0 | 100/0 | 100/0 | 100/0 |
| Azo (mg) | 0 | 0 | 101 | 101 | 80 |
| IPA (ml) | 0 | 0 | 100 | 100 | 100 |
| R Time (hour)/Temp | 35/70 | 35/70 | 26/60 | 21/60 | 37/60 |
| (° C.) | | | | | |
| Polymer Properties I.V. | 1.04 | 1.44 | 0.6 | 1.24 | 0.75 |
| M_{w} (kDa)¹ | >377² | 258 | 163 | 395 | 222 |
| Elongation (%) | >250 | >250 | >250 | >250 | >250 |

¹In DCM or otherwise as indicated.
²In THF.

TABLE 3B
Two-step Synthesis and Properties of Segmented VAc and BMA
Copolymers Using only BMA in the Second Step

| Example Number | 2F | 2G | 2H | 2I | 2J |
|---|---|---|---|---|---|
| Step 1 | | | | | |
| VAc/BMA (g/g) | 25/8 | 20/8 | 18/10 | 70/10 | 70/10 |
| Azo (mg) | 105 | 100 | 105 | 250 | 250 |
| IPA (ml) | 0 | 0 | 0 | 10 | 10 |
| R Time (hour)/Temp | 1/70 | 1/70 | 1/70 | 54/60 | 46/60 |
| (° C.) | | | | | |
| Step 2 | | | | | |
| BMA/VAc (g/g) | 22/0 | 20/0 | 22/0 | 120/0 | 120/0 |
| Azo (mg) | 0 | 0 | 0 | 80 | 80 |
| IPA (ml) | 0 | 0 | 0 | 12 | 20 |
| R Time (hour)/Temp | 35/70 | 35/70 | 35/70 | 30/60 | 23/60 |
| (° C.) | | | | | |
| Polymer Properties I.V. | 1.66 | 1.39 | 1.63 | 0.81 | 0.74 |
| M_{w} (kDa)¹ | 273 | 268 | 275 | 225 | 264 |
| Elongation (%) | >250 | >250 | >250 | >250 | >250 |

¹In DCM.

### EXAMPLES 3A AND 3B

### Two-Step Synthesis of Segmented Polymers of Ethyl Methacrylate with Two Other Comonomers

A segmented copolymer was synthesized in a two-step process using ethyl methacrylate (EMA) and two other comonomers. Reaction charge and conditions for the bulk preparation (no IPA was used) of a segmented copolymer of EMA with VAc and BMA are summarized in Table II for Example 3A. No IPA was used in the preparation. Example 3B is also described in Table II. It is based on EMA, ethoxyethyl methacrylate (ETOEMA) and BMA. The properties of the polymers in Examples 3A and 3B are also outlined in Table II.

TABLE 4
Two-Step Synthesis and Properties of Segmented Polymers of
Ethyl Methacrylate (EMA) with Two Other Comonomers

| Example Number | 3A | 3B |
|---|---|---|
| Step 1 | 20/15 | 20/7.5 |
| Added Monomers (g) | VAc/EMA | ETOEMA/EMA |
| Azo (mg) | 100 | 100 |
| R Time (hour)/Temp (° C.) | 2.5/70 | 0.7/70 |
| Step 2 | 7.5 | 22.5 |
| Added Monomer (g) | BMA | BMA |
| R Time (hour)/Temp (° C.) | 34/70 | 2/70 |
| Polymer Properties | | |
| I.V. | 1.84 | 1.57 |
| M_{w} (kDa)* | — | — |
| Elongation (%) | >250 | >250 |

*In DCM.

### EXAMPLES 4A-4E

### Synthesis of Segmented BMA and VAc Copolymers with at Least 90:10 BMA to VAc Comonomer Ratio

A segmented BMA-VAC copolymer with a BMA to VAc comonomer ratio of at least 90:10 was synthesized. A two-step synthetic scheme was used to prepare the copolymer of Example 4A and entailed 1) a brief first step of one copolymerization cycle associated with incomplete consumption of the comonomer; and 2) the addition of a second aliquot of Azo initiator at the beginning of the second copolymerization step. In Example 4B, the copolymerization was conducted in an extended single step using a high single dose of the Azo initiator. The synthesis of Examples 4C and 4D polymers entailed preparing a low molecular prepolymer of one type in the first step and adding a monomer mixture that was rich in BMA in the second step. In Example 4E, a slightly higher temperature was used in the second step. Reaction charges for the preparation of the copolymers of Examples 4A to 4E and their properties are summarized in Table III.

TABLE 5
Synthesis and Properties of BMA and VAc Segmented
Copolymers With at Least 90:10 BMA:VAc Comonomer Ratio

| Example Number | 4A | 4B | 4C | 4D | 4E |
|---|---|---|---|---|---|
| Step 1 | | | | | |
| VAc/BMA (g/g) | 10.5/200 | 20/180 | 15/0 | 0/195 | 15/0 |
| Azo (mg) | 250 | 300 | 200 | 200 | 200 |
| IPA (ml) | 100 | 100 | 100 | 150 | 100 |
| R Time (hr)/Temp | 10/65 | 21/65 | 16/65 | 12/65 | 16/65 |
| (° C.) | | | | | |
| Step 2 | | | | | |
| BMA/VAc (g/g) | 0/0 | 0/0 | 5/180 | 10.5/5 | 5/180 |
| Azo (mg) | 50 | 50 | 150 | 200 | 150 |
| R Time (hr)/Temp | 7/65 | 7/65 | 12/65 | 16/65 | 12/70 |
| (° C.) | | | | | |
| Polymer Properties | 1.08 | — | — | — | — |
| I.V. | | | | | |
| M_{w} (kDa)* | 314 | 183 | 260 | 300 | 310 |
| Elongation (%) | >250 | >250 | >250 | >250 | >250 |

*In DCM.

### EXAMPLES 5A-5D

### Synthesis of Segmented VAc and BMA Copolymers with at Least 90:10 VAc:BMA Comonomer Ratio

A segmented VAc and BMA copolymer with greater than 90:10 VAc to BMA comonomer ratio was synthesized in a two-step process. The two-step synthesis schemes outlined in Table IV were used to prepare the copolymers of Examples 5A and 5B. These entailed 1) charging all reactants at the first step; 2) using a relatively higher Azo concentration than those used in the previous examples; 3) extending the reaction time in the first step; and 4) limiting the reaction temperature in the second step to 25° C. For the copolymers of Examples 5C and 5D as in Table 6, the BMA was charged at both steps and an additional amount of IPA was used in the second step.

TABLE 6
Synthesis and Properties of VAc and BMA Segmented Copolymers
With at Least 90:10 VAc:BMA Comonomer Ratio

| Example | 5A | 5B | 5C | 5D |
|---|---|---|---|---|
| VAc/BMA (g/g) | 190/10 | 180/20 | 180/10 | 180/15 |
| Azo (mg) | 300 | 300 | 300 | 300 |
| IPA (ml) | 100 | 100 | 100 | 100 |
| R time (hour)/Temp (° C.) | 30/65 | 30/65 | 30/65 | 30/65 |
| Step 2 | | | | |
| BMA/VAc (g/g) | 0/0 | 0/0 | 0/10 | 0/5 |
| Azo (mg) | 0 | 0 | 0 | 0 |
| IPA (ml) | 0 | 0 | 100 | 100 |
| R time (hour)/Temp (° C.) | 12/25 | 12/25 | 12/65 | 12/65 |
| Polymer Properties | | | | |
| I.V. | — | 0.25 | — | — |
| M_{w} (kDa)* | — | 59 | — | — |
| Elongation (%) | >250 | >250 | >250 | >250 |

*In DCM.

### EXAMPLE 6

### Synthesis of Segmented VAc and BMA Copolymer with 50:50 VAc:BMA Comonomer Ratio

A segmented VAc and BMA copolymer with approximately 50:50 VAc:BMA comonomer ratio by weight was synthesized in a multi-step process. The multi-step synthetic scheme was used to prepare the 50:50 copolymer following the general experimental protocol as described in Example 1 for VAc and BMA copolymerization with the Azo initiator and IPA as a dilutent and reaction medium. In the first step, 85 g of VAc was allowed to copolymerize with 10 g of BMA using 200 mg of the Azo initiator and 75 ml of IPA. The polymerization was conducted at 65° C. for 24 hours. At the conclusion of the first step, a mixture of 5 g VAc, 30 g BMA, 50 mg Azo, and 25 ml IPA was added to the reaction product to proceed with the second step. At this step, the copolymerization was conducted at a temperature at about 65° C. for 6 hours. A similar charge was used in the third step, and the copolymerization was conducted at 65° C. for 16 hours. In the fourth step, the same comonomer, Azo and IPA charge was used, and the copolymerization was conducted at 65° C. for 16 hours. At the conclusion of the fourth step, the copolymer may be isolated, purified, and characterized in the manner as described in Example 1.

### EXAMPLE 7

### Synthesis of Segmented Interpenetrating VAc and BMA Copolymer with an Overall Comonomer Ratio of 50:50

A segmented copolymer consisting of interpenetrating VAc and BMA sequences with an average comonomer ratio of 50:50 by weight was synthesized. The polymerization was conducted in two steps under the general experimental conditions noted in earlier examples. In the first step, a mixture of 90 g VAc, 10 g BMA, 350 mg Azo, and 100 ml IPA was heated at 65° C. for 24 hours. At the conclusion of the first step, a mixture of 10 g VAc, 90 g MBA and 50 ml IPA was added to the reaction mixture, and heated to 65° C. and held there for 16 hours during the second step. The resulting polymer may be isolated, purified, and characterized as described in previous examples.

### EXAMPLE 8

### Synthesis of Segmented Interpenetrating VAc, nHMA and NVP Terpolymer with an Overall monomer Ratio of 30:40:30

A segmented terpolymer consisting of interpenetrating VAc, nHMA and NVP sequences with an average monomer ratio of approximately 30:40:30 by weight was synthesized as follows. However, it is understood that the following synthetic process can be used to synthesize numerous terpolymers have the same monomer constituents by with different monomer ratios merely by differing the relative concentrations of the starting materials in accordance with the teachings of the present invention.

Materials

a) 1-Vinyl-pyrrolidinone, 99+%, Aldrich catalog #V340-9 (L/N 08229KA), (vacuum distilled before use).
b) Vinyl acetate, 99+%, Aldrich catalog #V150-3 (L/N 03625DA)
c) n-Hexyl methacrylate, TCI America catalog #M0503 (UN GBO1)
d) 1,4-Dioxane, HPLC grade, 99.9 %, Aldrich catalog #27053-9 (L/N 02062DA)
e) 2,2′-Azobisisobutyronitrile (AJBN), Aldrich catalog #44109-0 (L/N 01313EA)
f) Hexanes, ACS reagent grade, 98.5 %, Aldrich catalog #44349-2 (L/N 07346HA)
g) Methanol, HPLC grade, Aldrich catalog #270474 (L/N 03935LA)
   Equipment
a) 500 mL Reaction kettle (VWR catlog #36390-020, Clamp VWR catalog #36393-030)
b) Stirring shaft (Chemglass catalog #CG-2079A-02)
c) Stirrer bearing (teflon) (Chemglass catalog #CG-2077-01)
d) Thermocouple (Controls Corp.)
e) HPLC delivery pump (Rabbit-HP HPX)
f) Balance (Meffler PM 4600)

g) Ace thread #7 with N2 inlet (Aceglass catalog #5261-16)

Formulation of Charges

| | Weight (g) |
|---|---|
| Charge One | |
| Vinyl acetate | 25 |
| 1-Vinyl pyrrolidone | 6.5 |
| n-Hexyl methacrylate | 8.5 |
| 1,4-Dioxane | 50 |
| AIBN | 0.375 |
| Charge Two | |
| 1-Vinyl pyrrolidone | 20.5 |
| Hexyl methacrylate | 40 |
| 1,4-Dioxane | 60 |
| AIBN | 0.45 |

Procedures
1) A 500 mL reaction kettle equipped with a mechanical stirrer (Teflon bearing and glass stirring shaft), a thermocouple¹ adapter with N₂ inlet an adapter for Charge Two addition tubing and a condenser capped with N₂ reaction bubbler, is charged with Charge One.
2) 20 % excess of Charge TWO is prepared and stored in a bottle. The Charge Two bottle is capped with a rubber septum thread with Teflon tubing, which is connected to a HPLC delivery pump. The tubing was filled with Charge Two solution. Charge Two bottle is purged with N₂ for 2 minutes².
3) The kettle is purged with N₂ at a flow rate of about a bubble/sec while under stirring for 20 minutes at room temperature. The N₂ bubbling is reduced just enough to maintain a positive pressure.
4) The reaction kettle is lowered to a preheated water bath (temperature set at about 62 C). The temperature should reach 60 C in about 5-10 minutes. The reaction is stirred at 60 C for 25 minutes before Charge Two is added at a rate of 20.16 g/hour. After 120.95 g of Charge Two is added (6 Hours), stop the HPLC pump and the reaction.
5) The reaction kettle is removed from the water bath and reaction is exposed to air and cooled to room temperature with an ice water bath.

The polymer solution is diluted with 2 L of hexanes and the solution is transferred to a flask. The polymer solution in hexanes is cooled to −60 C with a dry ice-isopropyl alcohol bath for 30 minutes to precipitate out the polymer. The system is warmed up to −40 C⁴. The top solution is decanted as much as possible. The sticky polymer is redissolved in 2L of hexanes. If the polymer does not completely dissolve at room temperature, warm it up with a water bath to raise the temperature to 50 C. Add just enough chloroform for the polymer to dissolve completely. The cold temperature precipitation is repeated two more times. The polymer is redissolved in 125 mL chloroform and precipitated in 1500 mL methanol cooled to −60 C. The solvent is decanted and the polymer is pressed and washed with some cold methanol. This precipitation is repeated one mote time. The sticky polymer is dissolved in 250 mL of chloroform and transferred to Teflon lined trays. After most of the chloroform is evaporated inside a hood, the polymer is dried in a vacuum oven set at 45 C under a vacuum of <1 mm Hg overnight. The transparent polymer film is peeled off the Teflon tray.

### EXAMPLE 9

### Synthesis of Segmented 94:5 VAc:BMA Copolymer Grafted with Short 1:20 VAc:BMA Chains, for an Overall Comonomer Ratio of 95:25 VAc:BMA

A segmented copolymer consisting of a 94:5 VAc:BMA copolymer grafted with short 1:20 VAc:BMA chains was synthesized in accordance with another embodiment of the present invention, to form a grafted copolymer with an overall comonomer ratio of 95:25. The polymerization was conducted in two steps, under the usual experimental conditions noted in earlier examples. In the first step, a mixture of 94 g VAc, 5 g BMA, 250 mg Azo, and 100 ml IPA was heated at 65° C. for 24 hours. At the conclusion of this period, 100 ml of IPA were added to the reaction product. This was followed by adding 50 mg of Azo and continuing the heating at 65° C. for 5 minutes prior to adding a mixture of 1 g VAc and 20 g BMA, and then proceeding with the second step of the copolymerization at 65° C. for 12 hours. The resulting polymer may be isolated, purified, and characterized as described in previous examples.

### EXAMPLE 10

### Preparation of Poly(n-Butyl Methacrylate)

In another embodiment of the current invention, a segmented copolymer was prepared with poly(n-butyl methacrylate). This entailed the use of 200 g BMA, 408 mg Azo initiator, and 150 ml IPA. The polymerization was conducted at 65° C. for 18 hours. The resulting polymer was isolated, purified, and characterized in the usual manner (see Example 1). The polymer was shown to have an l.V. of 0.82 dL/g, M_{w} (DCM) of 263 kiloDalton (kDa), and its film did not break at over 300% elongation.

As those skilled in the art will appreciate, the amount of cross-linking, monomer content, and molecular weight of the polymer effect the release of the nanoparticulate drug compounds. For example, if the polymer is highly cross-linked, the polymer can form a highly latticed network wherein the openings within the lattice are small. Thus, smaller particles that are associated with the polymer are able to pass through the openings while larger particles have a delayed release. Once the larger particles have been degraded by physical or chemical means into smaller sized particles, these particles can then pass through the openings of the polymer.

According to another embodiment of the present invention, the nanoparticulate compounds of the present invention can be suspended in a matrix. In one exemplary embodiment of the present invention, the matrix comprises openings having one single size. In another exemplary embodiment of the present invention, the matrix comprises openings having variable sizes. Like previous embodiments of the present invention, the size of the openings and the size of the drug compounds will determine the rate at which the compounds are released from the matrix. Thus, a drug releasing medical implant having controllable drug release can be achieved by varying the dimensions of the nanoparticles and the opening sizes.

Another aspect of the present invention is directed to methods of administering the nanoparticulate drug compounds of the present invention to a patient. According to one exemplary method, the nanoparticulate compounds are applied to a surface of a medical implant. The medical implant can then be delivered to a site of injury or disease. Subsequently, the nanoparticulate compounds can diffuse away from the implant surface to the site of injury or disease.

According to another exemplary method of the present invention, the release rate of the nanoparticulate compounds from a medical implant is regulated by the size of the openings on the implant surface and/or the size of the individual nanoparticulate compounds. That is, those particles small enough to pass through the openings on the implant surface are released. Those larger particles have a delayed release until the particles are small enough to pass through the openings. Thus, by varying the size of the nanoparticulate compounds and the openings on the implant surface, the release rate of the nanoparticulate compounds can be controlled.

For example, one exemplary method of the present invention is directed to minimizing restenosis. First, a medical implant such as, but not limited to, a self-expanding stent or a balloon catheter is coated with the nanoparticle coating of the present invention. The nanoparticle coating comprises nanoparticles of drug or anti-restenotic compounds such as, but not limited to, rapamycin. The medical implant is then delivered and deployed at the site of injury or disease. After implantation, the drug compounds diffuse from the surface of the implant into the surrounding environment and the walls of the vasculature.

Another exemplary method of the present invention is directed to treating aneurysms. More specifically, a medical implant such as, but not limited to, a self-expanding stent, a vascular graft, or a self-expanding stent graft is provided with a nanoparticle coating on at least one surface of the implant. The nanoparticle coating comprises compounds such as, but not limited to, matrix metalloproteinases inhibitors (MMPI). Exemplary MMPI include tetracycline, tricyclic sulfonamides, tricyclic heteroaromatics, and analogues and derivatives thereof as described in U.S. Pat. Nos. 6,420,408, 6,350,885, 6,265,432, and 6,169,103, which are hereby incorporated by reference. The medical implant is then subsequently delivered to the site of the aneurysm wherein the nanoparticulate compounds can be released into the surrounding environment and the walls of the vasculature.

Yet another exemplary method of the present invention is directed to treating vulnerable plaques. Vulnerable plaques are occlusions within the artery that can lead to acute clotting of the artery when exposed to appropriate triggers to events such as, but not limited to, ulceration rupture, erosion or thrombus. Once a vulnerable plaque is identified by techniques known and developed in the art, a medical implant having the nanoparticle coating of the present invention can be deployed at the site of the vulnerable plaque. According to one exemplary method, the medical implant can be a self-expanding stent. In one exemplary method, the nanoparticle coating comprises matrix metalloproteinase inhibitors having a particle sizes ranging from approximately 10 nm to approximately 1000 nm. In another exemplary method, the nanoparticle coating comprises dihydropyridine calcium channel blockers such as, but not limited to, lacidipine or amlodipine. Subsequently, the medical implant is deployed the site of the vulnerable plaque and the nanoparticle compounds associated with the nanoparticle coating can be released from the surface of the implant.

In closing, it is to be understood that the embodiments of the present invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely shown and described.

1. A nanoparticle coating system for medical implants comprising:
one or more drugs nanopulversized to have nanoparticulate sizes of approximately 10 nm to 500 nm and at least one biocompatible polymer.

2. The nanoparticle coating system of claim 1 wherein the drug is selected from the group consisitng of paclitaxel, docetaxel, epothilones, nitric oxide, heparin, aspirin, coumadin, PPACK, hirudin, polypeptide from angiostatin and endostatin, geldanamycin, herbimycin, macbecin, methotrexate, 5-fluorouracil, estradiol, P-selectin glycoprotein ligand-1 chimera, abciximab, exochelin, eleutherobin, sarcodictyin, fludarabine, sirolimus, rapamycin, ABT-578, certican, sulindac, tranilast, rosiglitazone, troglitazone, pioglitazone, darglitazone, englitazone, tetracyclines, VEGF, transforming growth factor (TGF)-beta, insulin-like growth factor (IGF), platelet derived growth factor (PDGF), fibroblast growth factor (FGF), RGD peptide, 17 beta-estradiol, radioactive agents and combinations thereof.

3. The nanoparticle coating system of claim 1 wherein the polymer is non-bioabsorbable a polymer.

4. The nanoparticle coating system of claim 1 wherein the polymer is bioabsorbable.

5. The nanoparticle coating of claim 1 wherein the drug is suspended in a matrix having a plurality of openings.

6. The nanoparticle coating of claim 5 wherein the openings have substantially similar sizes.

7. A medical implant comprising having a coating according to any one of claims 1 through 6.

8. The medical implant of claim 7 wherein the surface is a matrix having variable mesh size.

9. The medical implant of 7 wherein the surface is a matrix having single mesh size.

10. A controlled release coating for an implantable medical device comprising:
a terpolymer-bipolymer blend having a total solubility parameter (δ_{T}) approximately equal to a bioactive agent's solubility parameter (δ) and wherein δ_{T} and δ is between 15 J^{1/2}/cm^{3/2} to 25 J^{1/2}/cm^{3/2} and at least one drug agent nanopulversized to have nanoparticulate sizes of approximately 10 nm to 500 nm.

11. The controlled release coating according to claim 10 wherein said coating has a glass transition point (Tg) between approximately −20° C. and 50° C.

12. The controlled release coating according to claim 10 wherein said terpolymer comprises relative weight percent concentrations of monomer subunits consisting essentially of vinyl acetate (VAc), alkyl methacrylate (AMA) and n-vinyl pyrrolidone (NVP) and said bipolymer comprises relative weight percent concentrations of monomer subunits consisting essentially of VAc and AMA.

13. The controlled release coating according to claim 12 wherein said relative weight percent concentrations of said monomer subunits in said terpolymer comprises from 7-30% (VAc), 40-75% (AMA) and 19-30% (NVP).

14. The controlled release coating according to claim 12 wherein said relative weight percent concentrations of said monomer subunits in said bipolymer comprises from 5-70% VAc and from 30-95% AMA.

15. The controlled release coating according to claim 12 wherein said alkyl methacrylate is selected from the group consisting of methyl, ethyl, propyl, butyl, pentyl, and hexyl.

16. The controlled release coating according to any one of claims 10 though 15 wherein said δ_{T} is approximately 15 to 21 and said polymer blend comprises from 40% to 80% bipolymer and from 20% to 60% terpolymer.

17. The controlled release coating according to any one of claims 10 through 15 wherein said bipolymer has a lower Tg than said terpolymer.

18. The controlled release coating according to claim 1 wherein said drug is selected from the group consisting of anti-proliferatives including, but not limited to, macrolide antibiotics including FKBP 12 binding compounds, estrogens, chaperone inhibitors, protease inhibitors, protein-tyrosine kinase inhibitors, peroxisome proliferator-activated receptor gamma ligands (PPARγ), hypothemycin, nitric oxide, bisphosphonates, epidermal growth factor inhibitors, antibodies, antibiotics, proteasome inhibitors anti-sense nucleotides and transforming nucleic acids.

19. The controlled release coating according to claim 18 wherein said antiproliferative is a FKBP 12 binding compound.

20. The controlled release coating according to claim 19 wherein said FKBP 12 binding compound is a macrolide antibiotic.

21. A vascular stent comprising:
a structure comprising a material, said material having a coating thereon comprised of a hydrophobic polymer;
a terpolymer-bipolymer blend over said hydrophobic polymer wherein the difference between the solubility parameters of said terpolymer-bipolymer blend and said bioactive agent is no greater than 10 J^{1/2}/cm^{3/2} and the total solubility parameter (δ_{T}) of said bioactive agent-containing terpolymer-bipolymer blend is no greater than 25 J^{1/2}/cm^{3/2} and at least one one drug nanopulversized to have nanoparticulate sizes of approximately 10 nm to 500 nm.

22. The vascular stent according to claim 21 wherein said hydrophobic polymer is parylene or a parylene derivative.

23. The vascular stent according to claim 21 wherein said terpolymer comprises relative weight percent concentrations of monomer subunits consisting essentially of vinyl acetate (VAc), alkyl methacrylate (AMA) and n-vinyl pyrrolidone (NVP) and said bipolymer comprises relative weight percent concentrations of monomer subunits consisting essentially of VAc and AMA.

24. The vascular stent according to claim 23 wherein said relative weight percent concentrations of said monomer subunits in said terpolymer comprises from 7-30% (VAc), 40-75% (AMA) and 19-30% (NVP).

25. The vascular stent according to claim 23 wherein said relative weight percent concentrations of said monomer subunits in said bipolymer comprises from 5-70% VAc and from 30-95% AMA.

26. The vascular stent according to claim 23 wherein said alkyl methacrylate is selected from the group consisting of methyl, ethyl, propyl, butyl, pentyl, and hexyl.

27. The vascular stent according to anyone of claims 21 though 26 wherein said δT is approximately 15 to 21 and said polymer blend comprises from 40% to 80% bipolymer and from 20% to 60% terpolymer.

28. The vascular stent according to anyone of claims 21 though 27 wherein said bipolymer has a lower Tg than said terpolymer.

29. The vascular stent according to claim 21 wherein said drug is selected from the group consisting of anti-proliferatives including, but not limited to, macrolide antibiotics including FKBP 12 binding compounds, estrogens, chaperone inhibitors, protease inhibitors, protein-tyrosine kinase inhibitors, peroxisome proliferator-activated receptor gamma ligands (PPARγ), hypothemycin, nitric oxide, bisphosphonates, epidermal growth factor inhibitors, antibodies, antibiotics, proteasome inhibitors anti-sense nucleotides and transforming nucleic acids.

30. The vascular stent according to claim 29 wherein said antiproliferative is a FKBP 12 binding compound.

31. The vascular stent according to claim 30 wherein said FKBP 12 binding compound is a macrolide antibiotic.
